(19) United States
(12) Patent Application Publication
(10) Pub. No.: US 2008/0058317 A1
(43) Pub. Date: Mar. 6, 2008

(54) MILNACIPRAN FOR THE TREATMENT OF FATIGUE ASSOCIATED WITH FIBROMYALGIA SYNDROME

(75) Inventors: Srinivas G. Rao, Encinitas, CA (US); R. Michael Gendreau, Poway, CA (US); Jay D. Kranzler, La Jolla, CA (US)

Correspondence Address: DARBY & DARBY P.C., P.O. BOX 770, Church Street Station, New York, NY, 10008-0770, US

(73) Assignee: CYPRESS BIOSCIENCE, INC., San Diego, CA (US)

(21) Appl. No.: 11/835,590

(22) Filed: Aug. 8, 2007

Related U.S. Application Data
(60) Provisional application No. 60/836,857, filed on Aug. 9, 2006.

(51) Int. Cl. A61K 31/165; A61K 31/195; A61K 31/197; A61K 31/4168; A61K 31/428; A61K 31/433; A61K 31/48; A61K 31/485; A61K 31/496; A61K 31/522; A61K 31/5513; A61P 43/00

(52) U.S. Cl. 514/221; 514/254.06; 514/263.31; 514/282; 514/288; 514/362; 514/367; 514/401; 514/618; 514/620

(57) ABSTRACT

Methods for treating fatigue associated with fibromyalgia by administering high-dose milnacipran to a patient suffering from such fatigue are provided. Also provided are methods for the long-term treatment of fatigue associated with FMS by administering milnacipran to a patient suffering from such fatigue.

This application claims the benefit of U.S. provisional application No. 60/836,857, filed Aug. 9, 2006, the entire disclosure of which is incorporated by reference.

### FIELD OF THE INVENTION

The field of the invention relates to the treatment of fatigue associated with fibromyalgia by administering high-dose milnacipran to a patient suffering from fatigue associated with fibromyalgia.

### BACKGROUND

Fibromyalgia, also known as the fibromyalgia syndrome (FMS) is a common systemic rheumatologic disorder estimated to affect 2% to 4% of the population, second in prevalence among rheumatologic conditions only to osteoarthritis. Wolfe et al., Arthritis Rheum. 1990; 33(2):160-172; Wolfe et al., Arthritis Rheum. 1995; 38(1):19-28. Fibromyalgia is associated with a reduced threshold for pain, generally identified by an increased sensitivity to pressure all over the body, and is often accompanied by fatigue, sleep disturbance, and morning stiffness. Other common symptoms include headache, migraine, variable bowel habits, diffuse abdominal pain, and urinary frequency. The diagnostic criteria for fibromyalgia require not only a history of widespread pain, but also the finding of tenderness on physical examination (“tender points”). In order to fulfill the criteria for fibromyalgia established in 1990 by the American College of Rheumatology (ACR), an individual must have both widespread pain involving all four quadrants of the body as well as the axial skeleton, and the presence of 11 of 18 tender points on examination. Wolfe et al., Arthritis Rheum. 1990; 33(2):160-172.

While there has been some suggestion that FMS may represent a form of somatization disorder, there is increasing evidence and acceptance that FMS is a medical problem, reflecting a generalized heightened perception of sensory stimuli. The abnormality is thought to occur within the central nervous system (CNS) rather than peripherally, and the proposed pathophysiological defect is termed “central sensitization”. Clauw D J and Chrousos G P, Neuroimmunomodulation 1997; 4(3):134-153; Yunas M B, J Rheumatol. 1992; 19(6):846-850; Bradley et al., Curr Rheumatol Rep. 2000; 2(2):141-148; Simms R W, Am J Med. Sci. 1998; 315(6):346-350. FMS patients typically suffer from both allodynia (perceiving pain even from a non-painful stimulus such as light touch) and hyperalgesia (an augmentation of pain processing in which a painful stimulus is magnified and perceived with higher intensity than it would be by a normal volunteer). Mountz et al., Arthritis & Rheumatism 1995; 38(7):926-938; Arroyo J F and Cohen M L, J Rheumatol. 1993; 20(11):1925-1931. In this regard, there are many parallels in its clinical presentation and proposed underlying mechanisms with neuropathic pain, such as diabetic neuropathy and trigeminal neuralgia. Sindrup S H and T S Jensen, Pain 1999; 83(3):389-400; Woolf C J, Nature 1983; 306(5944):686-688; Woolf C J and R J Mannion, Lancet 1999; 353(9168):1959-1964. As a result, FMS is treated primarily within the medical model. It is most often diagnosed in the primary care setting, and almost half of the office visits are to internal medicine and family practice providers (1998 National Ambulatory Medical Care Survey). Visits to rheumatologists account for 16% of FMS patients' office visits. The remainder of visits are to a variety of tertiary care providers, including pain centers, physical medicine specialists, and psychiatrists.

Individuals with fibromyalgia suffer from a number of other symptoms, including a high incidence of recurrent non-cardiac chest pain, heartburn, palpitations, and irritable bowel syndrome. Wolfe, et al., Arthritis Rheum. 1990; 33(2):160-172; Mukerji et al., Angiology 1995; 46(5):425-430. Although the physiologic basis of these symptoms remains unclear, increasing evidence suggests that dysfunction of the autonomic nervous system is common in fibromyalgia and related illnesses. Clauw D J and Chrousos G P, Neuroimmunomodulation 1997; 4(3):134-153; Freeman R and Komaroff A L, Am J Med. 1997; 102(4):357-364. Prospective studies of randomly selected individuals with fibromyalgia have detected objective evidence of dysfunction of several visceral organs, including a 75% incidence of echocardiographic evidence of mitral valve prolapse, a 40-70% incidence of esophageal dysmotility, and diminished static inspiratory and expiratory pressures on pulmonary function testing. Lurie et al., Scand J Rehab Med. 1990; 22(3):151-155; Pellegrino et al., Arch Phys Med Rehab. 1989; 70(7):541-543. Neurally-mediated hypotension and syncope also appear to occur more frequently in individuals with fibromyalgia. Rowe et al., Lancet 1995; 345(8950):623-624.

Fibromyalgia is associated with high rates of disability, increased health care utilization, more frequent psychiatric consultations and a greater number of lifetime psychiatric diagnoses than controls.

A broad array of medications is used off-label in patients with FMS with varying degrees of success. Buskila D, Baillieres Best Pract Res Clin Rheumatol. 1999; 13(3):479-485; Leventhal L J, Ann Intern Med. 1999; 131(11):850-858; Lautenschlager J, Scand J Rheumatol Suppl. 2000:113:32-36. While antidepressants are the cornerstone of many treatment paradigms, other agents such as anti-convulsants, antispasticity agents, anxiolytics, sedatives, and opiates have been used. Non-steroidal anti-inflammatory drugs (NSAIDs) and acetaminophen are also used by a large number of patients (Wolfe et al., Arthritis Rheum. 1997; 40(9):1571-1579), even though peripheral inflammation has not been demonstrated (Clauw D J and Chrousos G P, Neuroimmunomodulation 1997; 4(3):134-153), and numerous studies have failed to confirm their effectiveness as analgesics in FMS. Goldenberg et al., Arthritis Rheum. 1986; 29(11):1371-1377; Yunus et al., J Rheumatol. 1989; 16(4):527-532; Wolfe et al., Arthritis Rheum. 2000; 43(2):378-385; Russell et al., Arthritis Rheum. 1991; 34(5):552-560; Quijada-Carrera et al., Pain 1996;65(2-3):221-225. These agents do, however, provide an element of protection against other peripheral pain generators, such as osteoarthritis.

Antidepressants of all varieties represent a common form of therapy for many chronic pain states, including FMS. Sindrup S H and Jensen T S, Pain 1999; 83(3):389-400; Buskila D, Baillieres Best Pract Res Clin Rheumatol. 1999; 13(3):479-485; Leventhal L J, Ann Intern Med. 1999; 131(11):850-858; Lautenschlager J, Scand J Rheumatol Suppl. 2000; 113:32-36; Bennett R M, J Functional Syndromes 2001; 1(1):79-92. The majority of available antidepressants directly and/or indirectly increase the levels of 5-HT and/or NE in the CNS. Monoaminergic levels are increased either by inhibiting re-uptake (by blocking transport proteins) or interfering with the breakdown of the monoamine (by inhibiting the monoamine oxidase enzymes) after its release into the synaptic cleft.

Tricyclic Antidepressants (TCAs)

The TCAs most commonly employed in the treatment of FMS include amitriptyline, doxepin, and cyclobenzaprine. Buskila D, Baillieres Best Pract Res Clin Rheumatol. 1999; 13(3):479-485; Lautenschlager J, Scand J Rheumatol Suppl. 2000; 113:32-36; Bennett R M, J Functional Syndromes 2001; 1(1):79-92. While cyclobenzaprine is typically classified as a muscle relaxant rather than an antidepressant, it shares structural and pharmacological similarities with the TCAs, although its sedating qualities often override its usefulness in other applications. Kobayashi et al., Eur. J. Pharmacol. 1996; 311(1):29-35. TCAs block the re-uptake of both 5-HT and NE, but they favor NE re-uptake blockade, and the efficacy of TCAs can be interpreted to support the primacy of NE agonism for analgesic activity. However, TCA's additional anti-cholinergic, antihistaminergic, and α-adrenergic receptor blockade activities impart a wide assortment of undesirable side effects, which often compromise their tolerability and clinical acceptance. Kent J M, Lancet 2000; 355(9207):911-918.

TCAs have demonstrated moderate efficacy for the treatment of neuropathic pain conditions such as post-herpetic neuralgia and painful diabetic neuropathy. Max et al., Neurology 1988; 38(9):1427-1432; Max et al., N Eng J Med. 1992; 326(19):1250-1256; Watson et al., Neurology 1982; 32(6):671-673; Watson et al., Pain 1992; 48(1):29-36. Multiple studies of TCAs in the treatment of FMS support their use for this syndrome as well, and TCAs have frequently been used as the positive controls to which newer agents have been compared. Max et al., N Eng J Med. 1992; 326(19):1250-1256; Watson et al., Pain 1992; 48(1):29-36; Hannonen et al., Br J Rheumatol. 1998; 37(12):1279-1286; Goldenberg et al., Arthritis & Rheumatism 1996; 39(11):1852-1859.

Selective Serotonin Re-Uptake Inhibitors (SSRIs)

The SSRIs have revolutionized the treatment of depression with their improved side-effect profile secondary to more selective re-uptake inhibition. The SSRI agents fluoxetine, sertraline and citolopram have each been evaluated in randomized, placebo controlled trials in FMS. Goldenberg et al., Arthritis & Rheumatism 1996; 39(11):1852-1859; Wolfe et al., Scand J Rheum. 1994; 23(5):255-259; Anderberg et al., Eur J Pain 2000; 4(1):27-35; Norregaard et al., Pain 1995; 61(3):445-449. However, the results of these trials have been somewhat inconsistent, leaving much debate regarding the relative efficacy of the SSRIs, especially in comparison to TCAs.

Two placebo-controlled trials of citalopram, the most 5-HT-specific of the SSRIs (see Table 2), in FMS patients were both convincingly negative. Anderberg et al., Eur J Pain, 2000; 4(1):27-35; Norregaard et al., Pain 1995; 61(3):445-449. This suggests that serotonergic enhancement alone is not sufficient to impart analgesia in the chronic pain setting. In fact, based on the evidence assembled to date, the SSRIs, as a class, are generally less efficacious than the TCAs in chronic pain states (Max et al., N Engl J Med. 1992; 326(19):1250-1256; Ansari A, Harv Rev Psych. 2000; 7(5):257-277; Atkinson et al., Pain 1999; 83(2):137-145; Jung et al., J Gen Intern Med. 1997; 12(6):384-389) although there are some exceptions (Saper et al., Headache 2001;41(5):465-474).

Dual Re-Uptake Inhibitors

Dual re-uptake inhibitors, referred to either as “SNRI's” or “NSRI's,” are pharmacologically similar to TCAs (such as amitriptyline and doxepin), exhibiting dual activity upon 5-HT and NE re-uptake. Sanchez C and Hytell J, Cell Mol Neurobiol. 1999; 19(4):467-489. However, these newer agents are generally devoid of significant activity at other receptor systems, resulting in diminished side effects and enhanced tolerability. Therefore, this class of antidepressant may have significant potential for the treatment of FMS and/or other chronic pain conditions. SNRIs that are commercially available in the U.S. include venlafaxine and duloxetine. A number of such agents are in clinical development; these include milnacipran, bicifadine, viloxazine, LY-113821, SEP-227162, AD-337, and desvenlafaxine succinate (DVS-233).

One small, open-label trial of venlafaxine (EFFEXOR®) in 15 patients with FMS showed promising results. Dwight et al., Psychosomatics 1998; 39(1):14-17. Six of 11 completing patients had a positive response to venlafaxine, defined as 50% or greater improvement in two different measurements of overall pain. Insomnia was the most common side effect reported, requiring adjunctive medical therapy in 3 of 11 completing patients.

U.S. Pat. No. 6,602,911 describes the use of milnacipran for the treatment of FMS and its symptoms, the entire disclosure of which is hereby incorporated by reference.

Opioids

Opiates exert their anti-nocioreceptive effects at various locations within both the ascending and descending pain pathways. Duale et al., Neuroreport 2001; 12(10):2091-2096; Besse et al., Brain Res. 1990;521(1-2):15-22; Fields et al., Nature 1983; 306(5944):684-686; Yaksh et al., Proc Natl Acad Sci USA 1999; 96(14):7680-7686. Concerns regarding the use of opioids in chronic pain conditions have been raised. Bennett R M, J Functional Syndromes 2001; 1(1):79-92. Opioids are used by some in the clinical management of FMS, especially when other analgesics have failed to provide sufficient relief. Bennett R M, Mayo Clin Proc. 1999; 74(4):385-398.

To date, there have been no published reports of effective, long-term treatment for fibromyalgia and its symptoms. Carette et al. reported the long-term (greater than three months) results of a clinical trial in which amitriptyline (a tricyclic antidepressant), cyclobenzaprine (a muscle relaxant structurally similar to tricyclic antidepressants) and placebo were administered to subjects suffering from fibromyalgia syndrome (Carette et al., Arthritis & Rheumatism 1994; 37(1):32-40). After one month, 21% of the amitryptyline subjects, 12% of the cyclobenzaprine subjects, and 0% of the placebo subjects had significant clinical improvement. At three months, there was no difference between either treatment group and placebo. At six months, no long-term efficacy could be demonstrated because of a higher than expected placebo response, i.e., 19% improvement with placebo.

Fatigue associated with fibromyalgia can lead to significant impairment of a patient's ability to carry out the activities of daily living, and the majority of patients with this fatigue remain symptomatic for years. Thus, a need exists for an effective, long-term treatment of fatigue associated with fibromyalgia syndrome.

### SUMMARY OF THE INVENTION

Surprisingly, according to the methods of the present invention, the administration of high dose milnacipran (e.g., more than about 125 mg/day) to a subset of FMS patients with fatigue associated with their FMS provides significantly more effective treatment for this fatigue than a dose of 100 mg/day milnacipran. This improved efficacy was unexpected because patients with pain associated with their FMS received about the same benefit from typical-dose (e.g., about 50 mg/day to about 100 mg/day) milnacipran compared to high dose milnacipran.

A double-blind, randomized, placebo-controlled clinical study (see Example 1 below) unexpectedly showed that administering high-dose milnacipran provides effective long-term (i.e., at least three months) treatment for fatigue associated with fibromyalgia in a patient suffering from such fatigue.

Until the discovery of the present invention, it was not known that patients with fatigue associated with fibromyalgia receive a greater benefit from high-dose (e.g., more than about 125 mg/day) milnacipran compared to a typical milnacipran dose of 100 mg/day. Conversely, it was known that high-dose milnacipran results in an adverse event profile, which is worse than the profile for typical milnacipran doses (e.g., 50 mg/day milnacipran or 100 mg/day milnacipran) (see, e.g., U.S. Publication No. 2004/0106681). Accordingly, prior to the present invention, physicians had no basis to recommend high-dose milnacipran to patients with fatigue associated with FMS. Moreover, prior to the present invention, physicians had no reason to identify patients with fatigue associated with FMS as a distinct subset of FMS patients because it was not known that such a subset of patients would benefit from a different dose of milnacipran relative to FMS patients generally.

In one aspect of the present invention, high-dose milnacipran provides effective, long-term treatment of fatigue associated with FMS for at least 3 months. In another aspect of the present invention, high-dose milnacipran provides effective, long-term treatment of fatigue associated with FMS for at least 6 months.

In certain embodiments of the present invention, high-dose milnacipran can be a dose of about 125 mg/day to about 400 mg/day. In other embodiments of the present invention, high-dose milnacipran can be a dose of about 150 mg/day to about 350 mg/day. In yet other embodiments of the present invention, high-dose milnacipran can be a dose of about 200 mg/day to about 300 mg/day. In further embodiments of the present invention, the dose of milnacipran is about 200 mg/day.

The present invention provides methods wherein the total amount (dose) of a high-dose milnacipran dosage can be administered once daily or in divided daily doses.

The present invention further provides methods for adjunctively administering a second active compound with milnacipran for the treatment of fatigue associated with FMS, wherein the second active compound is selected from the group consisting of: an antidepressant, an analgesic, a muscle relaxant, an anorectic, a stimulant, an antiepileptic drug, a beta blocker, and a sedative/hypnotic. In more particular embodiments, the second active compound for the treatment of fatigue associated with FMS is selected from the group consisting of: modafinil, gabapentin, pregabalin, pramipexole, 1-DOPA, amphetamine, tizanidine, clonidine, tramadol, morphine, tricyclic antidepressants, codeine, carbamazepine, sibutramine, valium, trazodone, caffeine, nicergoline, bifemelane, propranolol, and atenolol, and combinations thereof.

### BRIEF DESCRIPTION OF THE FIGURES

FIG. 1 is a timeline of the clinical study described in Example 1.

FIG. 2 is a bar graph which illustrates the percentage of FMS patients who responded to treatment of their pain associated with FMS at 3 months and 6 months for placebo, milnacipran 100 mg/day and milnacipran 200 mg/day groups.

FIG. 3 is a graph which shows that milnacipran 200 mg/day (“200”) is superior to milnacipran 100 mg/day (“100”) for the treatment of fatigue associated with FMS as measured by the MFI: physical fatigue. Both 200 and 100 are superior to placebo (“Pbo”) for the treatment of fatigue associated with FMS. Assessments were made at treatment weeks 3, 7, 11, 15, 19, 23 and 27.

FIG. 4 is a graph which illustrates the percentage of patients whose BDI (OC) “loss of energy” status changed from Tx0 to Tx15 in the clinical study described in Example 1.

FIG. 5 is a graph which illustrates the percentage of patients whose BDI (OC) “fatigue” status changed from Tx0 to Tx15 in the clinical study described in Example 1.

FIG. 6 is a Dose Escalation Flow Chart for the clinical study described in Example 2.

FIG. 7 is a timeline of the clinical study described in Example 2.

FIG. 8 is a graph which illustrates the percentage of patients whose BDI (OC) “loss of energy” status changed from Tx0 to Tx15 in the clinical study described in Example 2.

FIG. 9 is a graph which illustrates the percentage of patients whose BDI (OC) “fatigue” status changed from Tx0 to Tx15 in the clinical study described in Example 2.

### DETAILED DESCRIPTION

As used herein, the term “subject” or “patient” includes human and non-human mammals.

As used herein, “treatment” or “effective treatment” means relief from symptoms of fibromyalgia. More particularly, “treatment” or “effective treatment” of fatigue associated with FMS means relief of such fatigue. Relief of fatigue in a patient can be measured subjectively, e.g., a patient reports feeling less fatigued or tired, or objectively, e.g., the patient's MFI score improves relative to their baseline MFI: Physical Fatigue score.

As used herein, the term “high-dose” means a dose of at least about 125 milligrams (mg) per day. For example, in one embodiment, high-dose means about 125 mg to about 400 mg per day. In another embodiment, high-dose means about 200 mg to about 300 mg per day. In a more particular embodiment, high-dose means about 200 mg per day.

The terms “dual norepinephrine serotonin reuptake inhibitor” (NSRI) and “dual serotonin norepinephrine reuptake inhibitor” (SNRI) are synonymous and refer to a well-recognized class of anti-depressant compounds that selectively inhibit reuptake of both norepinephrine and serotonin. Common NSRI and SNRI compounds include, but are not limited to, venlafaxine, duloxetine, bicifadine and milnacipran.

The terms “NE>5-HT NSRI” and “NE>5-HT SNRI” are synonymous and refer to a subclass of NSRI compounds that inhibit norepinephrine reuptake more than or equal to serotonin reuptake. Milnacipran and bicifadine are examples of NE>5-HT NSRIs.

NSRI (SNRI) and NE>5-HT NSRI (NE>5-HT SNRI) compounds are described in detail in U.S. Pat. No. 6,602,911, the contents of which are hereby incorporated by reference.

According to the present invention, patients with fatigue associated with fibromyalgia syndrome (FMS) can be identified by a health care provider based on a FMS patient's chief complaint of, for example, fatigue, tiredness or the inability to carry out routine daily activities (e.g., household cleaning, errands) lasting >3 months in duration, or a Multidimensional Fatigue Inventory (MFI) score of 10 total or physical fatigue score of 8 or greater.

The MFI is a 20-item self-report instrument that measures 5 dimensions of fatigue; General Fatigue, Physical Fatigue, Mental Fatigue, Reduced Motivation and Reduced Activity (Smets et al., J Psychosom Res 1995, 39:315-325). The score in each dimension reflects the severity of fatigue (higher values indicate greater fatigue).

Milnacipran

Milnacipran is an NSRI, i.e., a dual noradrenaline and serotonin re-uptake inhibitor, exhibiting a novel chemical structure. Milnacipran is a CIS-(dl) racemate (Z form) composed of two (1- and d-) enantiomers. The chemical name of milnacipran's hydrochloride salt is: Z-2-aminomethyl-1-phenyl-N,N-diethylcyclopropanecarboxamide hydrochloride. Milnacipran's chemical formula is C₁₅H₂₃ Cl N₂O.

Adverse events associated with milnacipran administration include: nausea, vomiting, headache, tremulousness, anxiety, panic attack, palpitations, urinary retention, orthostatic hypotension, diaphoresis, chest pain, rash, weight increase, back pain, constipation, diarrhea, vertigo, increased sweating, agitation, hot flushes, fatigue, somnolence, dyspepsia, dysuria, dry mouth, abdominal pain, and insomnia. Due to the high incidence of adverse events, patients often do not tolerate high-dose milnacipran. The present invention encompasses the discovery that a particular group of patients with FMS, i.e., patients with fatigue associated with FMS, receive an unexpected benefit from the administration of high-dose milnacipran. Accordingly, for this group of patients, the benefit from high-dose milnacipran outweighs the potential detriment of one or more adverse event.

Milnacipran monotherapy for the treatment of fibromyalgia and/or symptoms associated with fibromyalgia was previously described in a Phase II trial of 125 fibromyalgia patients. See, e.g., co-pending U.S. application Ser. No. 10/678,767, the contents of which are hereby incorporated by reference in their entirety. In this study, milnacipran was administered once or twice daily in a dosage escalation regimen to a maximum dose of 200 mg/day. Treatment with milnacipran provided a wide range of beneficial effects on the signs and symptoms of FMS. Twice-daily (BID) and once-daily (QD) dosing of milnacipran were approximately equally effective on fatigue, mood, global wellness, and function. Twice-daily dosing was better tolerated than QD dosing, and was more effective in treating pain than QD dosing. The patient global impression of change (PGIC) outcome measure showed that over 70% of completers in both milnacipran treatment groups reported an improvement in their overall status, while only 10% reported worsening. In contrast, 40% of the placebo patients who completed the trial rated themselves as worse at endpoint. The differences between placebo and milnacipran on the PGIC were statistically significant, both in terms of a comparison of mean endpoint scores, as well as on a binary improved/not-improved basis.

Milnacipran was well-tolerated in this Phase II study. There were no deaths or serious adverse events (AEs) associated with milnacipran treatment, and the majority of AEs reported were rated as mild or moderate in severity. The most frequently reported AE was nausea, reported (one or more times) by 33% of milnacipran-treated patients; all other AES were reported in less than 9% of milnacipran-treated patients. The higher incidence of nausea, abdominal pain, headache and certain other AEs in the 200 mg QD treatment group suggests that larger doses taken once daily are not as well tolerated as smaller divided doses given twice a day. The reporting of dizziness, postural dizziness, hot flushes (and flushing), and palpitations was also greater in the QD treatment group, suggesting that peak drug level may be a significant factor in the generation of certain adverse effects.

Consistent with previous trial results, 7% of patients experienced mild increases in ALT and/or AST (<2 times the upper limit of normal), without concomitant increases in bilirubin or alkaline phosphatase. Elevation in hepatic enzymes resulted in adverse events in only 2% of milnacipran-treated patients (i.e., 2 out of 7 patients with enzyme elevations reported the adverse event of “elevation in SGOT” or “elevation in SGPT”).

A 4 to 8 beats-per-minute increase in mean heart rate was noted in milnacipran-treated patients, which was consistent with previous milnacipran trial results. Mean systolic and diastolic blood pressure among the milnacipran treatment groups showed only slight increases, ranging from 1.5 to 3.4 mmHg for supine systolic pressures (−1.1 to 2.7 mmHg in the placebo group), and 2.6 to 3.7 mmHg for supine diastolic pressures (−3.5 to 1.2 mmHg in the placebo group). Two (2%) milnacipran BID-treated patients reported an exacerbation of hypertension; both patients had pre-existing hypertension and were receiving antihypertensive drug therapy. One patient withdrew early from the trial due to an exacerbation of hypertension.

The potential for treatment-related orthostatic effects has also been documented during previous trials, and 6 (6%) of milnacipran-treated patients during the FMS trial reported the adverse event of orthostatic/postural dizziness, with one patient discontinuing early due to moderate postural dizziness. Vital sign data revealed that 4% of placebo patients and 7% of milnacipran patients experienced one or more visits with a decrease of 20 mm Hg or more in systolic blood pressure after standing erect for one minute.

Thus, this Phase II trial showed that treatment with 100 mg BID milnacipran was an effective acute (short-term) therapy for the symptom of pain in FMS, and milnacipran dosed either once or twice daily had measurable beneficial effects on a wide range of symptoms of FMS, including fatigue (measured on the FIQ), pain (multiple measures), quality of life (multiple measures), and, potentially, mood (Beck instrument).

Effective Dosages:

Pharmaceutical compositions suitable for use in the present invention include high-dose milnacipran and a pharmaceutically acceptable carrier or excipient. The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and, more particularly, in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin, the entire disclosure of which is hereby incorporated by reference.

In some embodiments of the present invention, milnacipran is administered in a dose of between about 125 mg/day and about 400 mg/day. In other embodiments, milnacipran is administered in a dose of between about 150 mg/day and about 350 mg/day. In yet other embodiments, milnacipran is administered in a dose of between about 200 mg/day and about 300 mg/day. In some embodiments, milnacipran is administered in a dose of about 200 mg/day.

The route of administration of a pharmaceutical composition of the present invention can be, for example, oral, enteral, intravenous, and transmucosal (e.g., rectal). A preferred route of administration is oral.

Pharmaceutical compositions suitable for oral administration can be in the form of tablets, capsules, pills, lozenges, powders or granules, or solutions or dispersions in a liquid. Each of said forms will comprise a predetermined amount of a compound of the invention as an active ingredient. The composition in the form of a tablet can be prepared employing any pharmaceutical excipient known in the art for that purpose, and conventionally used for the preparation of solid pharmaceutical compositions. The examples of such excipients are starch, lactose, microcrystalline cellulose, magnesium stearate and binders, for example polyvinylpyrrolidone. Furthermore, an active compound can be formulated as controlled-release preparation, such as tablets comprising a hydrophilic or hydrophobic matrix.

A pharmaceutical composition of the present invention can be in the form of a capsule formulated using conventional procedures, for example by incorporation of a mixture of an active compound and excipients into a hard gelatin capsule. Alternatively, a semi-solid matrix of an active compound and high molecular weight polyethylene glycol can be formed and filled into hard gelatin capsules, or soft gelatin capsules can be filled with a solution of an active compound in polyethylene glycol or dispersion thereof in an edible oil. Powder forms for reconstitution before use (for example lyophilized powders) are also contemplated. Alternatively, oily vehicles for injection formulation can be used as well.

Liquid forms for parenteral administration can be formulated for administration by injection or continuous infusion.

Accepted routes of administration by injection are intravenous, intraperitoneal, intramuscular and subcutaneous. A typical composition for intravenous injection comprises a sterile isotonic aqueous solution or dispersion, including, for example, an active compound and dextrose or sodium chloride. Other examples of suitable excipients are lactated Ringer solution for injections, lactated Ringer solution for injections with dextrose, Normosol-M with dextrose, acylated Ringer solution for injections. The injection formulation can optionally include a co-solvent, for example polyethylene glycol, chelating agent, for example ethylenediaminotetraacetic acid; stabilizing agent, for example cyclodextrin; and antioxidant, for example sodium pyrosulfate.

The high-dose milnacipran dosage may be administered once per day or in divided doses that are given two or more times per day. The amount of milnacipran administered to practice the methods of the present invention can vary depending on the subject being treated, the severity of the affliction, the manner of administration and the judgment of the prescribing physician.

Combination Therapy:

According to the present invention, milnacipran can be administered adjunctively with other active compounds for the long-term treatment of fatigue associated with FMS. Other active compounds according to the invention include, for example, antidepressants, analgesics, muscle relaxants, anorectics, stimulants, antiepileptic drugs, beta blockers, and sedative/hypnotics. Specific examples of compounds that can be adjunctively administered with the NE 5-HT SNRI compounds include, but are not limited to, modafinil, gabapentin, pregabalin, pramipexole, 1-DOPA, amphetamine, tizanidine, clonidine, tramadol, morphine, tricyclic antidepressants, codeine, carbamazepine, sibutramine, valium, trazodone, propranolol, atenolol, and combinations thereof. In an embodiment of the present invention, milnacipran is adjunctively administered with an alpha-2-delta ligand such as, for example, pregabalin or gabapentin.

As used herein, adjunctive administration includes simultaneous administration of the compounds in the same dosage form, simultaneous administration in separate dosage forms, and separate administration of the compounds. For example, milnacipran can be simultaneously administered with valium, wherein both milnacipran and valium are formulated together in the same tablet. Alternatively, milnacipran can be simultaneously administered with valium, wherein both the milnacipran and valium are present in two separate tablets. In another alternative, milnacipran can be administered first followed by the administration of valium, or vice versa.

The following examples are merely illustrative of the present invention and should not be construed as limiting the scope of the invention in any way as many variations and equivalents that are encompassed by the present invention will become apparent to those skilled in the art upon reading the present disclosure.

### EXAMPLES

### Example 1

### A Multi-Center Double-Blind, Randomized, Placebo-Controlled Study of Milnacipran for the Treatment of Fibromyalgia

The primary objective of this study was to demonstrate safety and efficacy, both clinical and statistical, of milnacipran in the treatment of the fibromyalgia syndrome. The primary outcome was a composite responder analysis assessing response rate at weeks 14 and 15, and the secondary analysis assessed response rate at weeks 26 and 27.

Other objectives of this study were to:

1. compare statistical and clinical efficacy of 100 mg/day and 200 mg/day milnacipran in the treatment of the fibromyalgia syndrome based on each component of the composite responder analysis, as well as on a number of additional secondary endpoints including fatigue, sleep and mood, and cognition; and

2. establish and compare the safety profiles of 100 and 200 mg milnacipran daily in patients with FMS.

Methodology

This was a multi-center, randomized, double-blinded, placebo-controlled three-arm study, which enrolled 888 patients who met the 1990 ACR criteria for fibromyalgia syndrome as well as the more detailed admission criteria outlined in the protocol.

Patients recorded baseline symptoms for the first two weeks after washing off anti-depressants, benzodiazepines, and certain other drugs that could potentially interfere with efficacy measurements.

Patients were randomized to receive either placebo, 100 mg/day milnacipran, or 200 mg/day milnacipran in a ratio of a 1:1:2. All randomized medications (placebo and milnacipran) were administered in a split-dose (BID) fashion. The doses were administered in a dose escalation regimen as outlined below:

Step 1: 12.5 mg 1 day (12.5 mg pm)

Step 2: 25 mg 2 days (12.5 mg am, 12.5 mg pm)

Step 3: 50 mg 4 days (25 mg am, 25 mg pm)

Step 4: 100 mg 7 days (50 mg am, 50 mg pm)

Step 5: 200 mg 7 days (100 mg am, 100 mg pm)

All patients were scheduled to receive a total of 24 weeks of milnacipran or placebo after the 3 weeks of dose escalation steps, for a total of 27 weeks of milnacipran or placebo exposure.

Patients were required to complete electronic diary, as well as additional paper assessments as described in the schedule of study assessments.

Adverse event, physical examination, concomitant medication, vital sign and clinical laboratory data were collected as detailed in the schedule of study assessments.

Patients who successfully completed this double blind trial were eligible to participate in an open label trial for 15 to 28 additional weeks of therapy.

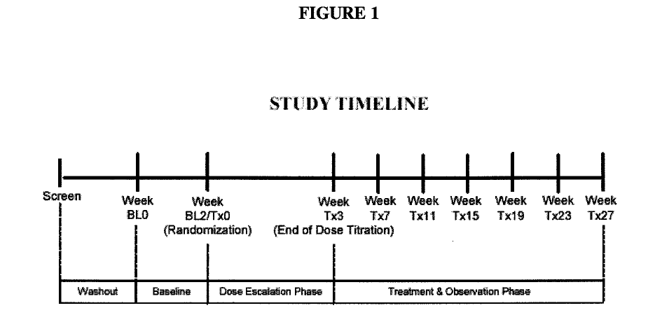

A timeline of the study is provided in FIG. 1.

Assessments

Safety:

Safety of milnacipran was assessed by analyzing the frequency and severity of adverse events, changes in vital signs and clinical laboratory data collected during the study period.

Efficacy:

In addition to the daily completion of a proprietary electronic patient diary, the following assessments were obtained:

a. Primary Variables: patient global impression of change (PGIC) and the Fibromyalgia Impact Questionnaire (FIQ).

b. Psychological Screening at baseline: M.I.N.I.

c. Miscellaneous status assessments: periodically, as described in the schedule of evaluations: BDI, sleep quality scale, and the ASEX.

d. FMS Status Assessments: Patient pain 24 hour and 7 day recall VAS, the SF-36, Multiple Ability Self-report Questionnaire (MASQ, cognitive function), the Multidimensional Health Assessment Questionnaire (MDHAQ) and the Multidimensional Fatigue Inventory (MFI). Diary assessments include current pain (morning, random daily, and evening reports); daily recall pain (morning report); medications taken (evening report); overall pain past week (weekly report), overall fatigue in the last week (weekly report), and the extent that pain kept the patient from caring for themselves (weekly report).

The SF-36 is a multi-purpose, short-form health survey. It yields an 8-scale profile of functional health and well-being scores, psychometrically-based physical and mental health summary measures, and a preference-based health utility index (Ware J E, Snow K K, Kosinski M, Gandek B. SF-36® Health Survey Manual and Interpretation Guide. Boston, Mass.: New England Medical Center, The Health Institute, 1993). The SF-36 provides a measure of a patient's functional impairment due to fatigue (i.e., how fatigue affects daily living activities of a patient). The SF-36 has proven useful in surveys of general and specific populations, comparing the relative burden of diseases, and in differentiating the health benefits produced by a wide range of different treatments.

The MASQ is a brief self-report questionnaire, which includes 5 cognitive domains: language ability, visuo-perceptual ability, verbal memory, visual memory, and attention/concentration (Seidenberg et al., J Clin & Exp Neuropsychology 1994; 16:93-104). The MASQ has been validated in both normal subjects and patient groups having cognitive difficulties in the assessment domains.

Statistical Analysis

Efficacy:

The primary endpoint of this study was a composite responder analysis implementing analysis of three domains of interest, evaluated at 24 weeks as the primary analysis, and 12 weeks as the secondary analysis. The domains measured were:

1) pain (measured by an electronic diary as a daily recall pain score, calculated to weekly average scores)

2) patient global (measured by the PGIC, 1-7 scale)

3) physical function (measured by the FIQ-PF).

For the primary analysis, the pain domain score was determined by a calculation that compared the average of treatment weeks 14 and 15 to the two baseline weeks, and treatment weeks 26 and 27 vs. baseline for the secondary analysis. The last observation was carried forward if neither the week 14 nor week 15 (or week 26/27) patient self-reported pain score is available to compare to the baseline value.

The binary response rate for placebo (based on the composite endpoint) in this study was expected to be in the range of 10-13%, with a milnacipran response rate in the active arm(s) expected in the 27-29% range on an ITT/LOCF basis. Based on these response rate assumptions, 125 patients randomized per arm (250 for high dose group) has been calculated to be the maximum sample size required (90% power). Secondary analyses included total area under the curve of pain intensity, and patient-reported weekly pain recall at the clinic visits as well as the FMS status assessments, and QOL measures.

Results

A responder was defined as a subject who experienced a greater than 30% reduction in pain from baseline and improvement on the PGIC.

At three months, the percentage of responders was: 35.44% ( 56/158) in the placebo group; 53.33% ( 72/135) (p=0.001) in the milnacipran 100 mg/day group; and 55.00% ( 143/260) (p<0.001) in the milnacipran 200 mg/day group. At six months, the percentage of responders was: 32.86% ( 46/140) in the placebo group; 49.59% ( 60/121) (p=0.002) in the milnacipran 100 mg/day group; and 51.74% ( 119/230) (p<0.001) in the milnacipran 200 mg/day group. See Table 1 for a summary of the results in the Intent-to-Treat Population and Table 2 for a summary of the Last Observation Carried Forward (LOCF), Baseline Observation Carried Forward (BOCF) and study completer (OC) populations. LOCF is an analysis in which observations are carried forward to the last time point for patients who dropped out. The LOCF analysis treats the carried-forward data as observed data at the last time point. BOCF is an analysis that requires the patient remain active in the trial to be evaluated for response. If a patient withdraws from the trial for any reason they are classed as a non-responder irregardless of their pain and global scores at the time of withdrawal.

TABLE 1
Analysis of Responders for the Treatment of the Pain of Fibromyalgia during
Treatment Weeks 14-15 and 26-27 (Observed Cases) Intent-to-Treat Population

| | | Placebo | Milnacipran | Milnacipran |
|---|---|---|---|---|
| | Statistic | (N = 223) | 100 mg (N = 224) | 200 mg (N = 441) |
| Baseline pain | n | 223 | 224 | 441 |
| | mean | 68.37 | 68.32 | 69.41 |
| | SD | 11.98 | 11.54 | 11.85 |
| | SEM | 0.80 | 0.77 | 0.56 |
| | median | 66.5 | 67.9 | 69.1 |
| | min, max | 50, 100 | 41, 100 | 47, 99 |
| Treatment | n | 158 | 135 | 260 |
| weeks 14-15 | m (% = m/n) | 56 (35.44) | 72 (53.33) | 143 (55.00) |
| | odds ratio | | 2.10 | 2.20 |
| | 95% CI | | (1.31, 3.36) | (1.46, 3.31) |
| | p-value | | 0.002 | <0.001 |
| Treatment | n | 140 | 121 | 230 |
| weeks 26-27 | m (% = m/n) | 46 (32.86) | 60 (49.59) | 119 (51.74) |
| | odds ratio | | 1.96 | 2.20 |
| | 95% CI | | (1.18, 3.26) | (1.42, 3.41) |
| | p-value | | 0.009 | <0.001 |

TABLE 2
Summary of Composite Responder Rate
Pain Composite Resp

| | 3 month | | | 6 month | | |
|---|---|---|---|---|---|---|
| | pbo | 100 mg | 200 mg | pbo | 100 mg | 200 mg |
| | N = 223 | N = 224 | N = 441 | N = 223 | N = 224 | N = 441 |
| Primary | 27.8% | 33.5% | 34.9% | 25.1% | 30.8% | 32.2% |
| Analysis | | p* = 0.187 | p* = 0.058 | | p* = 0.197 | p* = 0.053 |
| (LOCF) | | | | | ap* = 0.393 | ap* = 0.105 |
| Sensitivity | 25.1% | 32.1% | 32.4% | 20.6% | 26.8% | 27.0% |
| Analysis I | | p* = 0.094 | p* = 0.048 | | p* = 0.167 | p* = 0.067 |
| (BOCF) | | | | | ap* = 0.334 | ap* = 0.133 |
| Sensitivity | 25.56% | 32.% | 32.7% | 21.5% | 27.2% | 28.6% |
| Analysis II | | p* = 0.113 | p* = 0.056 | | p* = 0.197 | p* = 0.048 |
| | | | | | ap* = 0.394 | ap* = 0.095 |
| Sensitivity | 25.1% | 32.1% | 32.4% | 22.9% | 29.5% | 29.9% |
| Analysis | | p* = 0.094 | p* = 0.048 | | p* = 0.120 | p* = 0.051 |
| III | | | | | ap* = 0.241 | ap* = 0.102 |
| OC | n* = 158 | n = 135 | n = 260 | n = 140 | n = 121 | n = 230 |
| Analysis | 35.4% | 53.3% | 55.0% | 32.9% | 49.6% | 51.7% |
| | | p* = 0.002 | p* < 0.001 | | p* = 0.009 | p* < 0.001 |

Summary of Individual Component Responder Rate

| | Pain Composite Resp, 3 month | | | Pain Composite Resp, 6 month | | |
|---|---|---|---|---|---|---|
| | pbo | 100 mg | 200 mg | pbo | 100 mg | 200 mg |
| Primary | 27.8% | 33.5% | 34.9% | 25.1% | 30.8% | 32.2% |
| Analysis | | p* = 0.187 | p* = 0.058 | | p* = 0.197 | p* = 0.053 |
| (LOCF) | | | | | ap* = 0.393 | ap* = 0.105 |
| Pain | 31.4% | 35.7% | 38.3% | 28.7% | 35.7% | 35.4% |
| (LOCF) | | p = 0.321 | p = 0.068 | | p = 0.110 | p = 0.072 |
| PGIC | 47.1% | 54.0% | 50.6% | 46.2% | 49.6% | 49.9% |
| (LOCF) | | p = 0.143 | p = 0.397 | | p = 0.476 | p = 0.368 |

*p-value: nominal p-value. ap = adjusted p-value at Step 2 for Hochberg's procedure (only valid if p-value is =<0.05 for 3-month pain for 200 mg compared to placebo at Step 1). n = number of patients having adequate date for OC analysis (completers of landmark endpoint with observed values for responder assessment).

These results surprising establish that continued administration of milnacipran (e.g., daily administration for at least three months) to subjects suffering from fibromyalgia provides long-term (at least three months) relief from fibromyalgia and its symptoms.

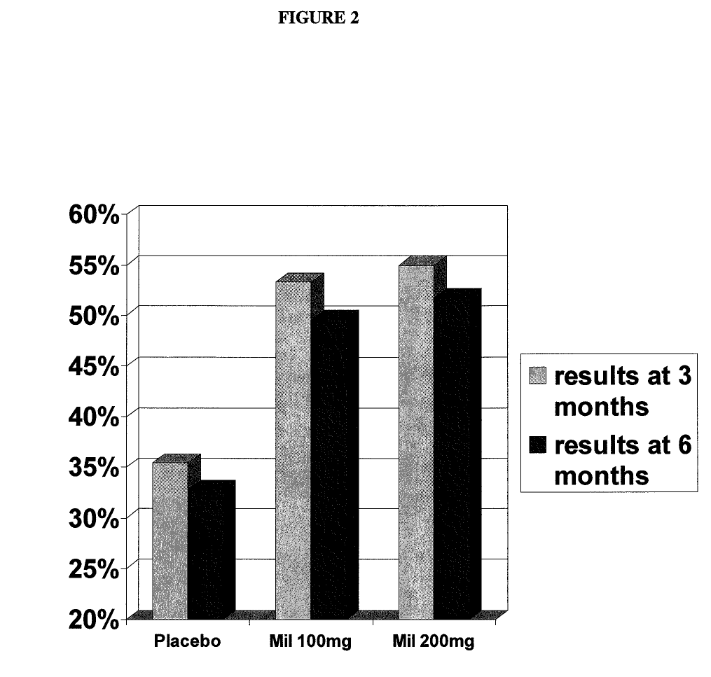

Further, these results surprisingly establish that continued administration of low dose milnacipran (e.g., 100 mg/day) is almost as effective as continued administration of high dose milnacipran (e.g., 200 mg/day) for the long-term treatment of fibromyalgia and some of its symptoms. FIG. 2.

The SF-36 Physical Function results are summarized in Table 3:

TABLE 3

| | | Milnacipran 100 | Milnacipran |
|---|---|---|---|
| | Placebo | mg/day | 200 mg/day |
| Treatment week | N = 223 | N = 224 | N = 441 |
| 3 | 4.32 | 6.31 | 7.44 |
| 7 | 5.23 | 6.62 | 8.80 |
| 11 | 6.23 | 7.70 | 8.46 |
| 15 | 5.32 | 7.70 | 8.44 |
| 19 | 4.63 | 7.71 | 8.37 |
| 23 | 5.70 | 7.07 | 7.85 |
| 27 | 5.77 | 7.11 | 7.95 |

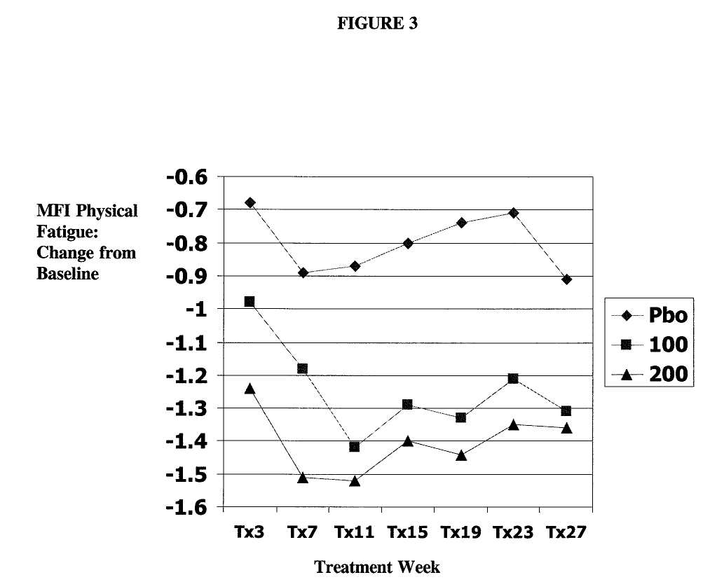

The results from the MFI total score and the MFI physical fatigue measurement change from baseline (summarized in Table 4) showed that milnacipran 100 mg/day and milnacipran 200 mg/day were superior to placebo for the treatment of fatigue associated with FMS. See FIG. 3. Moreover, these results establish that milnacipran 200 mg/day is superior to milnacipran 100 mg/day for the treatment of fatigue associated with FMS.

The Beck Depression Inventory contains two items that assess fatigue; namely, questions 15 (“Loss of Energy”) and 20 (“Tiredness or Fatigue”). Question 15 is scored from 0-3 (0=I have as much energy as ever, 1=I have less energy than 1 used to have, 2=I don't have enough energy to do very much, 3=I don't have enough energy to do anything). Question 20 is scored from 0-3 (0=I am no more tired or fatigued than usual, 1=I get more tired, or fatigued more easily, than usual, 2=I am too tired or fatigued to do a lot of the things I used to do, 3=I am too tired or fatigued to do most of the things I used to do).

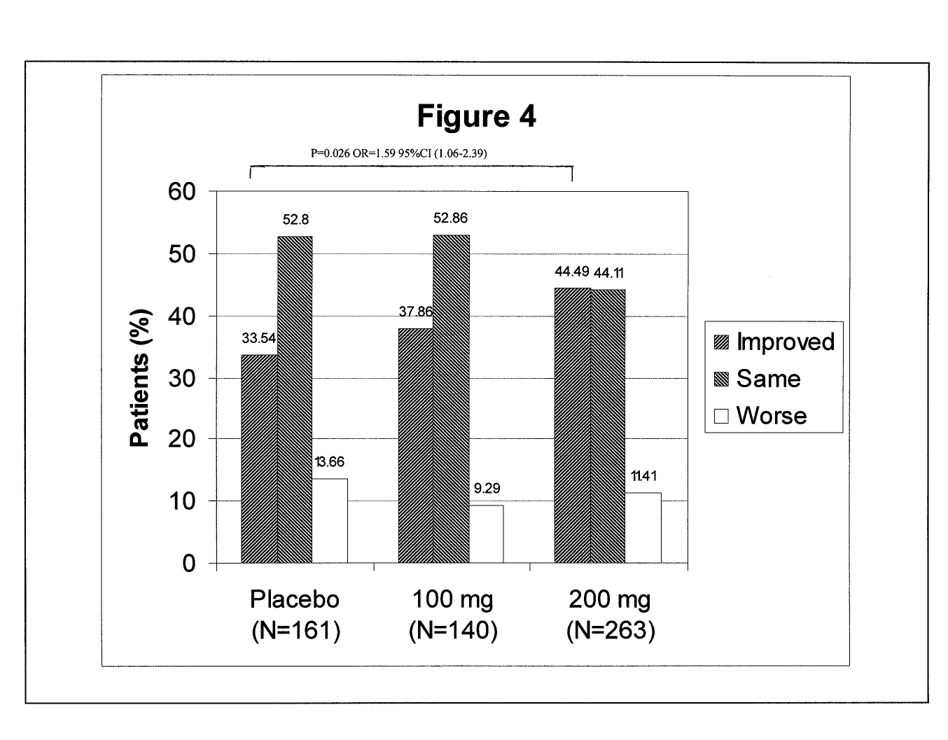

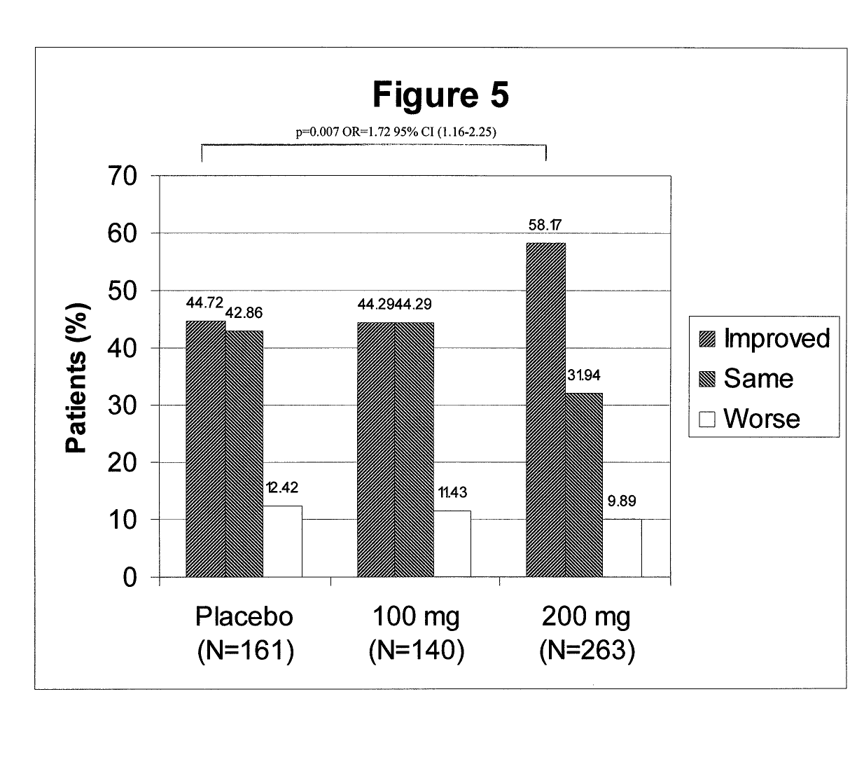

The percentage of patients whose BDI (OC) scores for “loss of energy” changed when administered 100 mg/day milnacipran, 200 mg/day of milnacipran or placebo are shown in FIG. 4. The percentage of patients whose BDI (OC) scores for “tiredness or fatigue” changed when administered 100 mg/day milnacipran, 200 mg/day of milnacipran or placebo are shown in FIG. 5.

TABLE 4
MFI (change from baseline)

| | Total score (range 20 to 100) | | | Physical fatigue (range 4 to 20) | | |
|---|---|---|---|---|---|---|
| Treatment | pbo | 100 mg | 200 mg | pbo | 100 mg | 200 mg |
| week | N = 223 | N = 224 | N = 441 | N = 223 | N = 224 | N = 441 |
| 3 | −2.34 | −3.68 | −4.58 | −0.58 | −0.99 | −1.20 |
| | | p = 0.123 | p = 0.014 | | p = 0.059 | p = 0.012 |
| 7 | −3.44 | −4.32 | −5.92 | −0.91 | −1.23 | −1.56 |
| | | p = 0.411 | p = 0.015 | | p = 0.119 | p = 0.009 |
| 11 | −3.43 | −5.16 | −5.67 | −0.85 | −1.46 | −1.54 |
| | | p = 0.116 | p = 0.041 | | p = 0.010 | p = 0.008 |
| 15 | −3.04 | −5.15 | −5.62 | −0.88 | −1.29 | −1.50 |
| | | p = 0.042 | p = 0.016 | | p = 0.065 | p = 0.019 |
| 19 | −2.60 | −4.97 | −5.67 | −0.81 | −1.34 | −1.54 |
| | | p = 0.026 | p = 0.006 | | p = 0.022 | p = 0.008 |
| 23 | −2.35 | −4.44 | −4.94 | −0.77 | −1.23 | −1.44 |
| | | p = 0.043 | p = 0.020 | | p = 0.035 | p = 0.013 |
| 27 | −3.35 | −5.00 | −5.80 | −1.01 | −1.37 | −1.49 |
| | | p = 0.098 | p = 0.035 | | p = 0.078 | p = 0.087 |

### Example 2

### A Multicenter, Double-Blind, Randomized, Placebo-Controlled Monotherapy Study of Milnacipran for Treatment of Fibromyalgia

The primary objective of this study was to demonstrate the safety and efficacy, both clinical and statistical, of milnacipran in the treatment of fibromyalgia syndrome (FMS) or the pain associated with fibromyalgia. The primary outcome was a composite responder analysis assessing response rates of two doses (100 mg/day and 200 mg/day) of milnacipran as compared with placebo at Visit Tx15 (week 15).

Secondary objectives were (i) to compare statistical and clinical efficacy of 100 mg/day and 200 mg/day of milnacipran with placebo in the treatment of FMS, based on the time-weighted average of each component outcome of the composite responder endpoint from Visits Tx3 to x15 and (ii) to establish and compare the safety profiles of 100 mg/day and 200 mg/day milnacipran in patients with FMS.

Methodology

This was a multicenter, randomized, double-blind, placebo-controlled three-arm study designed which enrolled 1196 patients who meet the 1990 ACR criteria for fibromyalgia syndrome (history of widespread pain and pain in 11 of 18 tender point sites on digital palpation), as well as the more detailed admission criteria outlined in the protocol.

Patients recorded baseline symptoms for the first two weeks after washing off anti-depressants, benzodiazepines, and certain other drugs that could potentially interfere with efficacy measurements.

Patients were randomized to receive placebo, 100 mg/day milnacipran or 200 mg/day milnacipran in a ratio of 1:1:1 (placebo=401 patients, 100 mg/day=399 patients, 200 mg/day=396 patients). The patients assigned to the two active treatment arms received a total of 12 weeks of stable-dose milnacipran exposure after the 3 weeks of dosage escalation steps, for a total of 15 weeks of drug exposure. All randomized medications (placebo and milnacipran) were administered twice a day (BID).

For the dose escalation period (Visits BL2/Tx0-Tx3), three blister cards were supplied, one for each week. On day one, in the evening, all three arms of the study received one large and one small capsule. In the case of the two active arms, the dose consisted of an active 12.5 mg capsule plus a placebo. In the case of the placebo arm, the dose consisted of one small and one large placebo capsule. On days two and three, the active arms each received one 12.5 mg active capsule plus a placebo capsule morning and evening and the placebo arm received two placebo capsules each morning and evening. For days 4-7, the active arms received one 25 mg active capsule plus a placebo capsule morning and evening and the placebo arm received 2 placebo capsules each morning and evening.

During the second week of the dose escalation period (i.e., days 8 through 14), patients in all three arms received only the larger 50 mg size capsules. Placebo patients received two large placebo capsules each time they take medication. Both the 100 mg and 200 active patients received one placebo and one active 50 mg capsule, morning and evening.

During the third week of the dose escalation phase, the placebo patients continued to receive two large placebo capsules, morning and evening. The 100 mg patients continued to receive one 50 mg active and one 50 mg placebo capsule, morning and evening. At this point, the 200 mg patients began receiving two 50 mg active capsules, morning and evening.

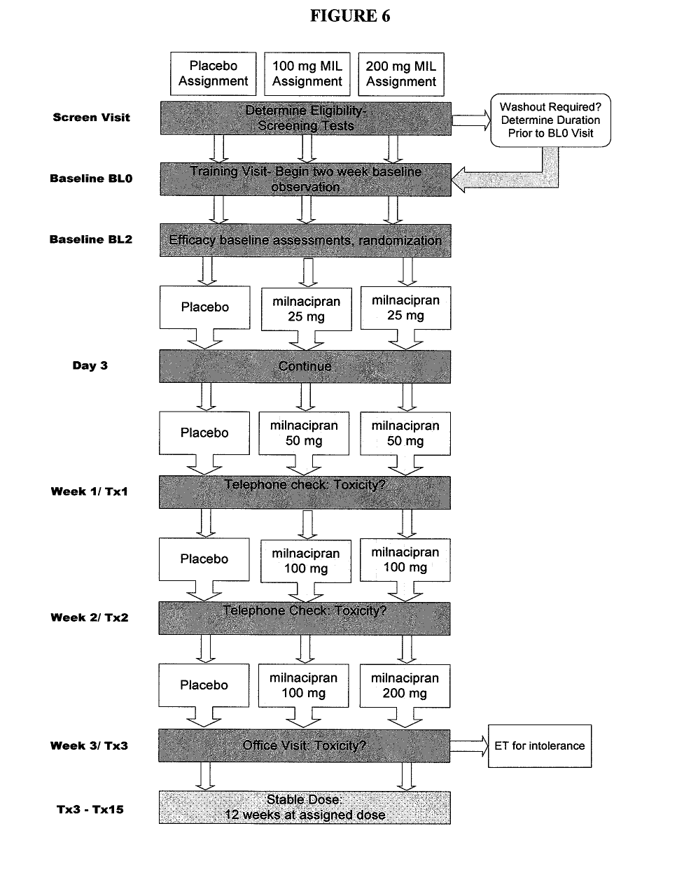

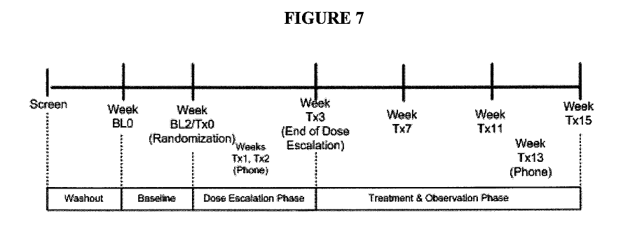

The dose escalation flow chart is shown in FIG. 6. A timeline of the study is provided in FIG. 7.

Patients were required to complete a proprietary electronic diary recording self-reported pain data as well as additional paper assessments as described in the schedule of study assessments.

Adverse event, physical examination, concomitant medication, vital sign, electrocardiogram (ECG) and clinical laboratory data were collected as detailed in the schedule of study assessments.

Assessments

Safety

Safety of milnacipran was assessed by analyzing the frequency and severity of adverse events (AEs), changes in vital signs, physical examination results, ECG, and clinical laboratory data collected during the study period.

Efficacy

In addition to the daily completion of an electronic diary system, the following assessments were obtained:

(i) Primary Efficacy Assessments: Patient Global Impression of Change (PGIC) administered to patients at visits Tx3, Tx7, Tx11 and Tx15/ET; Physical Component summary of SF-36 (SF-36 PCS) administered to patients at visits BL2/Tx0, Tx3, Tx7, Tx11 and Tx15/ET;

(ii) Secondary Efficacy Assessments: Time weighted average (AUC) of weekly average PED morning recall pain score; PGIC and SF-36 PCS administered to patients at visits Tx3 to Tx15.

(iii) Additional Efficacy Measurements: The Fibromyalgia Impact Questionnaire (FIQ) total score and physical function, Beck Depression Inventory (BDI), the MOS-Sleep Index Scale, the Arizona Sexual Experiences Scale (ASEX), Patient pain 24 hour and 7 day recall VAS, the SF-36 individual domains, Patient Global Disease Status, Patient Global Therapeutic Benefit, the Multiple Ability Self-report Questionnaire (MASQ, cognitive function), the Multidimensional Health Assessment Questionnaire (MDHAQ), Multidimensional Fatigue Inventory (MFI), and diary assessments including current pain (morning, random daily, and evening reports); overall pain past week (weekly report), overall fatigue in the last week (weekly report), and the extent that pain kept the patient from caring for themselves (weekly report).

The primary efficacy assessment of this study was a composite responder status defined by three domains of interest evaluated at visit Tx15. The domains measured were

1) pain (measured by an electronic diary in the morning as a daily recall pain score);

2) patient global (measured by the PGIC, 1-7 scale);

3) physical function (measured by the SF-36 PCS).

The primary efficacy parameter for an indication in the treatment of pain of fibromyalgia was the composite responder status based on the morning recall pain as recorded in the PED and patient global as recorded on the PGIC at Visit Tx15.

The primary efficacy parameter for an indication in the treatment of FMS was the composite responder status based on two domains of pain and patient global as used above in the primary efficacy parameter for the treatment of the pain of fibromyalgia plus the additional domain of physical function as measured by the SF-36 PCS at Visit Tx15.

The secondary efficacy parameters were time-weighted average (AUC) of the weekly average PED morning recall pain scores for Weeks 4 through 15, PGIC, and SF-36 PCS for Visit Tx3 to Visit Tx15.

The physical function domain for response analysis was measured by the Physical Component Summary of SF-36 (SF-36 PCS). The SF-36 is a brief, well-established, self-administered patient questionnaire for the assessment of health status, functional status, and quality of life. The SF-36 measures eight domains of health status: physical functioning, role limitations due to physical problems, bodily pain, general health perceptions, energy/vitality, social functioning, role limitations due to emotional problems, and mental health. An SF-36 PCS score and a mental component summary (MCS) score can be calculated by combining and weighting the various individual scales. The PCS and MCS scores have been standardized to have a mean=50, SD=10 in the general healthy US population (see, e.g., Ware, J., M. Kosinski, and J. Dewey, How to Score Version 2 of the SF-36 Health Survey (Standard & Acute Forms). 3rd ed. 2000, Lincoln, R.I.: QualityMetric).

Results

A patient was classified as a responder for the treatment of pain of fibromyalgia if he or she reached Visit Tx15 and satisfied the following criteria:
Greater than or equal to 30% in pain reduction from baseline;
PGIC rated as “much or very much improved,” (i.e., a score of 1 or 2 on the 1-7 scale at endpoint.)

A patient was classified as a responder for the treatment of FMS if he or she satisfied the responder criteria for the treatment of pain of fibromyalgia and the following additional criterion (at visit Tx15):
Improvement on the SF-36 PCS score from baseline by an amount at least equivalent to the minimal clinically important difference, as defined in the Statistical Analysis Plan.

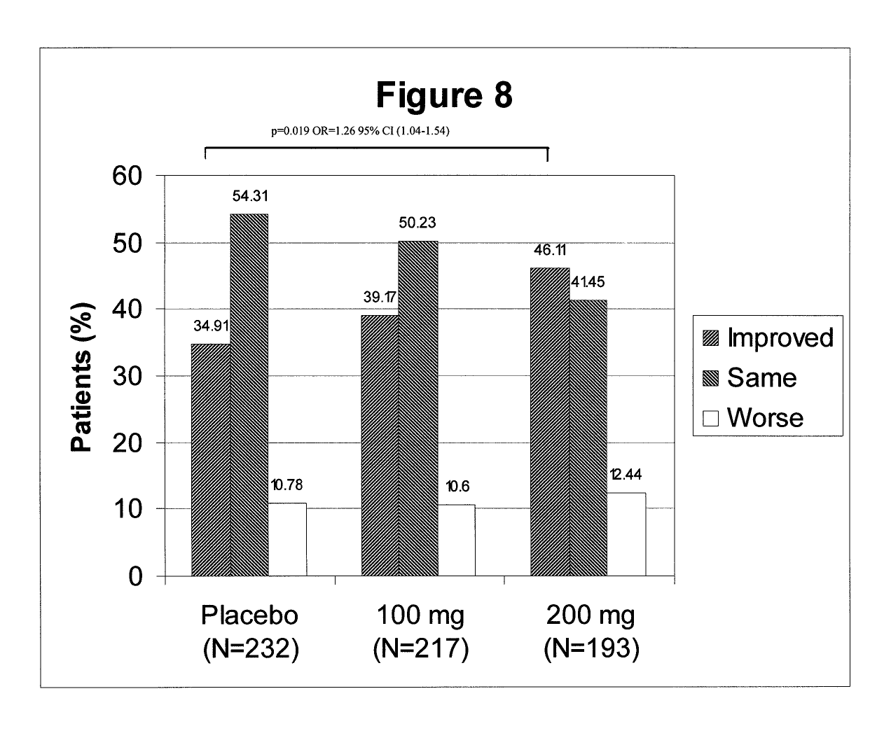

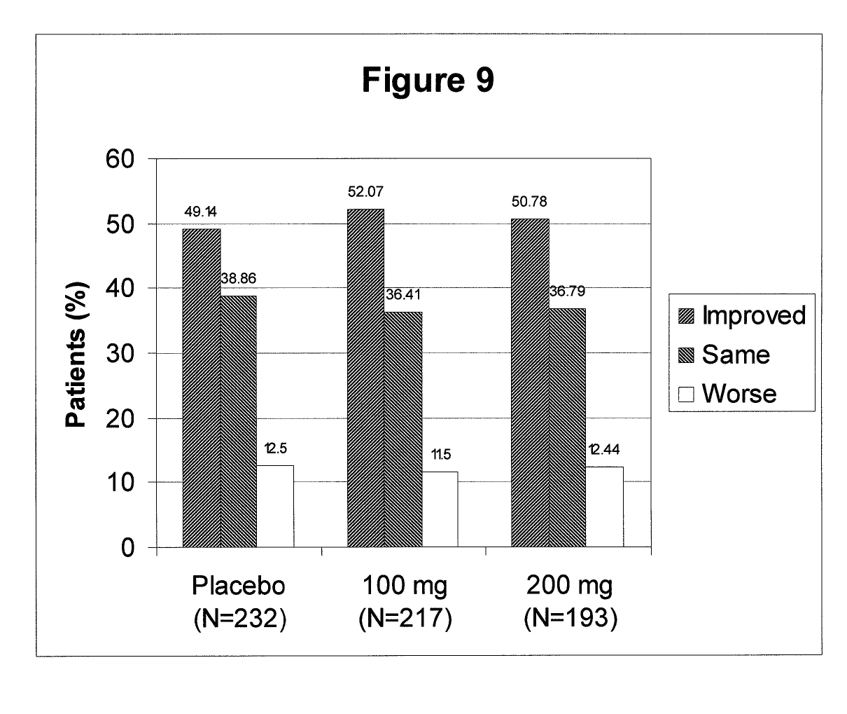

The percentage of patients whose BDI (OC) scores for “loss of energy” changed when administered 100 mg/day milnacipran, 200 mg/day of milnacipran or placebo are shown in FIG. 8. The percentage of patients whose BDI (OC) scores for “tiredness or fatigue” changed when administered 100 mg/day milnacipran, 200 mg/day of milnacipran or placebo are shown in FIG. 9.

Table 5 shows the change from baseline in the MFI total score by visit for the 3-month treatment period (LOCF), intent-to-treat population.

TABLE 5

| | Placebo | | 100 mg milnacipran | | 200 mg milnacipran | |
|---|---|---|---|---|---|---|
| | Actual | Change | Actual | Change | Actual | Change |
| n | 401 | 401 | 399 | 399 | 396 | 396 |
| Mean | 65.58 | −3.84 | 63.00 | −5.39 | 64.15 | −5.39 |
| SD | 14.85 | 12.00 | 15.91 | 12.77 | 14.77 | 12.13 |
| SEM | 0.74 | 0.60 | 0.80 | 0.64 | 0.74 | 0.61 |
| Median | 66.0 | −3.0 | 64.0 | −4.0 | 65.0 | −4.0 |
| Min, Max | 23, 100 | −59, 34 | 20, 97 | −54, 25 | 22, 99 | −58, 23 |
| LS Mean | | −4.08 (0.763) | | −5.47 (0.792) | | −5.63 (0.787) |
| (SE)* | | | | | | |
| Difference | | | | −1.80 | | −1.55 |
| from | | | | | | |
| Placebo* | | | | | | |
| 95% CI | | | | (−3.47, −0.13) | | (−3.17, 0.07) |
| P-Value* | | | | 0.035 | | 0.061 |

SD = standard deviation, SEM = standard error of the mean, min = minimum, max = maximum;
*Analyses for comparison to placebo are based the values of change from baseline (change) using an ANCOVA model with treatment group and study center as factors and baseline value as covariate. LS Mean and SE for the placebo group are from the model comparing 200 mg group with placebo

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects. All references cited herein are hereby incorporated by reference in their entirety.

1. A method of treating a fatigue symptom associated with fibromyalgia syndrome (FMS) comprising administering more than about 125 mg per day of milnacipran to a patient in need thereof.

2. The method of claim 1 wherein the fatigue is the primary symptom of FMS.

3. The method of claim 1, wherein the milnacipran is administered once daily.

4. The method of claim 1, wherein the milnacipran is administered in divided doses.

5. The method of claim 1, wherein the milnacipran is administered for at least 3 months.

6. The method of claim 1, wherein the milnacipran is administered for at least 6 months.

7. The method of claim 1, which comprises adjunctively administering a second active compound for the treatment of fatigue associated with FMS, wherein the second active compound is selected from the group consisting of an antidepressant, an analgesic, a muscle relaxant, an anorectic, a stimulant, an antiepileptic drug, a beta blocker, and a sedative/hypnotic.

8. The method of claim 6, wherein the second active compound for the treatment of fatigue associated with FMS is selected from the group consisting of gabapentin, pregabalin, pramipexole, 1-DOPA, amphetamine, tizanidine, clonidine, tramadol, morphine, tricyclic antidepressants, codeine, carbamazepine, sibutramine, valium, trazodone, atenolol, propranolol, and combinations thereof.

9. The method of claim 1, wherein between about 125 mg per day and about 400 mg per day of milnacipran is administered to the patient.

10. The method of claim 1, wherein between about 150 mg per day and about 350 mg per day of milnacipran is administered to the patient.

11. The method of claim 1, wherein between about 200 mg per day and about 300 mg per day of milnacipran is administered to the patient.

12. The method of claim 1, wherein about 200 mg per day of milnacipran is administered to the patient.
